(19) United States
(12) Patent Application Publication
(10) Pub. No.: US 2010/0227346 A1
(43) Pub. Date: Sep. 9, 2010

(54) Activation Mixture

(76) Inventor: Anthony Hubbard, Potters Bar (GB)

Correspondence Address: FROMMER LAWRENCE & HAUG, 745 FIFTH AVENUE- 10TH FL., NEW YORK, NY, 10151, US

(21) Appl. No.: 12/652,017

(22) Filed: Jan. 4, 2010

Related U.S. Application Data
(62) Division of application No. 11/280,473, filed on Nov. 16, 2005, now abandoned.
Application No. 11/280,473 is a continuation-in-part of application No. PCT/GB2004/002152, filed on May 17, 2004.

(30) Foreign Application Priority Data
May 16, 2003 (GB) 0311331.3
Nov. 13, 2003 (GB) 0326525.3

(51) Int. Cl. C12Q 1/56

(52) U.S. Cl. 435/13

(57) ABSTRACT

The present invention relates to an activation mixture comprising factor-deficient substrate plasma, an activator, and a phospholipid. The present invention also relates to a method for preparing, and the use of, an activation mixture. The present invention further relates to an assay method for determining the amount of blood coagulation factor in a test sample and a kit for performing the assay method described therein.

### FIELD OF INVENTION

The present invention relates to blood coagulation factors.

In particular, the present invention relates to an activation mixture for the measurement of blood coagulation factors.

### BACKGROUND TO THE INVENTION

The process of blood coagulation involves a series of proteins known as blood coagulation proteins which act in a cascade fashion to effect the formation of a blood clot. Haemophilia is a disease of humans and other mammals wherein a gene encoding a blood coagulation factor contains a mutation such that the encoded protein does not function normally in the cascade process.

Haemophilia A is the most common form of the disorder and is an X-linked, recessive, bleeding disorder caused by a deficiency in the activity of coagulation Factor VIII. Affected individuals develop a variable phenotype of hemorrhage into joints and muscles, easy bruising, and prolonged bleeding from wounds. The disorder is caused by heterogeneous mutations in the Factor VIII gene which maps to Xq28. Despite the heterogeneity in Factor VIII mutations, carrier detection and prenatal diagnosis can be done by direct detection of selected mutations (especially the inversions), as well as indirectly by linkage analysis. Replacement of Factor VIII is done using a variety of preparations derived from human plasma or recombinant techniques. While replacement therapy is effective in most cases, 10 to 15% of treated individuals develop neutralising antibodies that decrease its effectiveness. The mainstay of routine treatment for haemophilia A is infusion of Factor VIII done using amounts that are required to restore the Factor VIII activity to therapeutic levels. Since the half-life of Factor VIII is 8-12 hours twice daily infusions may be required in some circumstances.

The hereditary disease, haemophilia B, is characterised by a mutation in the gene encoding the blood coagulation protein, Factor IX (F.IX). F.IX is reviewed in High et al. (1995, “Factor IX” In: Molecular Basis of Thrombosis and Hemostasis, High and Roberts, eds., Marcel Dekker, Inc.).

In 1936, Patek and Stetson (J. Clin. Invest. 15, 531-542) reported that haemophilia could be corrected by the replacement of a plasma factor. Since this time, it has been necessary to have an assay for this factor (now called Factor VIII) to support clinical diagnosis, for monitoring treatment and for quality control of therapeutic Factor VIII preparations.

Various in vitro assays for this purpose have been developed. For example, a well known one-stage assay has been reported by Langdell et al. (1953) J. Lab. Clin. Med. 41, 637-647 and Hardisty and Macpherson (1962) Thromb. Diath. Haemorrh 7, 215-229 and a two stage assay has been reported by Biggs et al. (1955) Br. J. Haematol. 1, 20-34. Although other types of assay have been developed since this time, such as the chromogenic assay (Seghatchian and Miller-Anderson, (1978) Med. Lab. Sci. 35, 347-354) or fluorogenic assays (Mitchell et al. (1981) Thromb. Res. 21, 573-584), the classic assay systems are still in use.

Of all the kinds of assays for blood coagulation factors—such as Factor VIII—the one stage assay is the most commonly used for reasons, of speed, simplicity and ease of automation (Barrowcliffe et al., (1981) Haemostasis 11, 96-101). The principle of this assay is described by Over (1984) Scandinavian Journal of Haematology Supplementum No. 41, 33 13-24. The assay is based on the ability of Factor VIII-containing samples to correct the coagulation of Factor VIII-deficient plasma. By addition of a diluted Factor VIII-containing sample, the coagulation time is reduced, and as long as the dilution is chosen such that the amount of Factor VIII added is rate limiting, this shortening is a function of the amount of Factor VIII added. At high concentrations of Factor VIII, other factors become rate limiting whilst at low—concentrations the clotting times approach the longest clotting times. However, in between these extremes, a range of clotting times is usually present where the dose response is linear. By comparing the dose-response curve of an unknown sample with that of a sample with known activity (i.e. a standard), the Factor VIII content of the test sample can be determined.

Over (1984) Scandinavian Journal of Haematology Supplementum No. 41, 33 13-24. describes the procedure of the one stage assay as follows:

The test is performed by pipetting in turn a volume of Factor VIII-deficient substrate plasma, diluted test sample, phospholipid suspension, and activator reagent (the latter two may be added as a combined reagent). The mixture is then incubated at 37° C. The coagulation reaction is started by adding calcium ions and from this point on, the time to reach the endpoint is recorded.

Various modifications to the one-stage assay have been described in order to overcome problems associated with the reproducibility of the assay. Geiger et al. (1955) Proc. V. Congr. Europ. Soc. Haemat. Freiburg i. B., S. 413. Springer, Berlin added normal serum to the system. Waller (1959) Scand. J. Clin. Lab. Invest. 11, 194 attempted to improve the assay by careful standardisation of glassware and pre-incubation of the plasma mixture for 6 minutes before recalcification. Egeberg (1961) Scand. J. Clin. Lab. Invest. 13, 140 found that the measures described by Waller (1959) in addition to vigorous stirring of the substrate haemophilic plasma immediately before use also increased reproducibility. Hardisty & Macpherson (1962) Thromb. Diath. Haemorrh 7, 215-229 have described a further modification in which plasma is incubated with an optimal amount of kaolin immediately before recalcification.

The present invention relates to improvements in the one stage assay for determining the amount of blood coagulation factor in a sample.

### SUMMARY OF THE INVENTION

The present invention is based in part upon the surprising finding that a single activation mixture can be used in place of the individual components that are used in the existing one stage assay method.

Advantageously, the modified one stage assay method described herein, is quicker, easier to perform and may be easier to automate.

In a first aspect, the present invention relates to an activation mixture comprising factor-deficient substrate plasma, an activator, and a phospholipid.

In a second aspect, the present invention relates to a method for preparing an activation mixture comprising the step of mixing together factor-deficient substrate plasma, an activator and a phospholipid.

In a third aspect, the present invention relates to an assay method for determining the amount of blood coagulation factor in a test sample comprising adding an activation mixture to the test sample.

The blood coagulation factor may be measured for various applications—such as to diagnose or monitor treatment for haemophilia (e.g. to detect the specific cause of excessive bleeding), for carrier detection, during surgery, or as part of a quality control of therapeutic concentrates.

In a fourth aspect, the present invention relates to a kit for determining the amount of blood coagulation factor in a test sample comprising a first vessel containing an activation mixture.

In a fifth aspect, the present invention relates to the use of an activation mixture in an assay method for determining the amount of blood coagulation factor in a sample.

Preferably, the activator is micronised silica.

Preferably, the phospholipid is synthetic phospholipid.

Preferably, the activator and phospholipid are provided as a single reagent. More preferably, the single reagent is APTT reagent.

Preferably, the factor-deficient substrate plasma is chemically depleted or immuno depleted.

Preferably, the activation mixture is stable at 4° C. and/or 22° C. for at least 5 hours.

Preferably, the activator and the phospholipid are provided as a single reagent. More preferably the single reagent is APTT reagent.

Preferably, the assay method according to the third aspect of the present invention comprises the steps of: (a) providing a test sample; (b) providing an activation mixture according to any one of claims 1 to 7; (c) adding together the test sample and the activation mixture; (d) adding a calcium reagent; (e) measuring the clotting time/clotting end point; and (f) determining the amount of blood coagulation factor in the test sample.

Preferably, each of the assay components recited in steps (a), (b) and (d) are pre-heated. More preferably, each of the assay components recited in steps (a), (b) and (d) are pre-heated to about 37° C.

Preferably, the clotting time/clotting end point is measured using a coagulometer.

Preferably, the clotting time/clotting end point is measured using a point of care device and/or near patient care device.

Preferably, the kit comprises an additional vessel containing a calcium reagent.

Preferably, the kit comprises an additional vessel containing one or more blood coagulation factors.

Preferably, the kit comprises a point of care device or near patient care device.

Preferably, the activation mixture is preheated. More preferably, the activation mixture is preheated to about 37° C.

Other aspects of the present invention are presented in the accompanying claims and in the following description and discussion. These aspects are presented under separate section headings. However, it is to be understood that the teachings under each section heading are not necessarily limited to that particular section heading.

### ADVANTAGES

The present invention has a number of advantages. These advantages will be apparent to in the following description.

By way of example, the present invention is advantageous since it provides an assay method for determining the amount of blood coagulation factor in a sample that is faster to perform than existing methods.

By way of further example, the present invention is advantageous since it provides an assay method that comprises fewer steps than existing methods and is therefore simpler to perform.

By way of further example, the present invention is advantageous since it provides an assay method that may be easier to automate.

By way of further example, the present invention is advantageous since the reagents used in the assay method can be tailored specifically for estimation of the blood coagulation factor of interest.

### DESCRIPTION OF THE FIGURES

FIG. 1

Outline of the existing one-stage assay method and the modified one-stage assay method according to the present invention.

FIG. 2

A graph illustrating the stability of the activation mixture at 4° C. over 5 hours.

FIG. 3

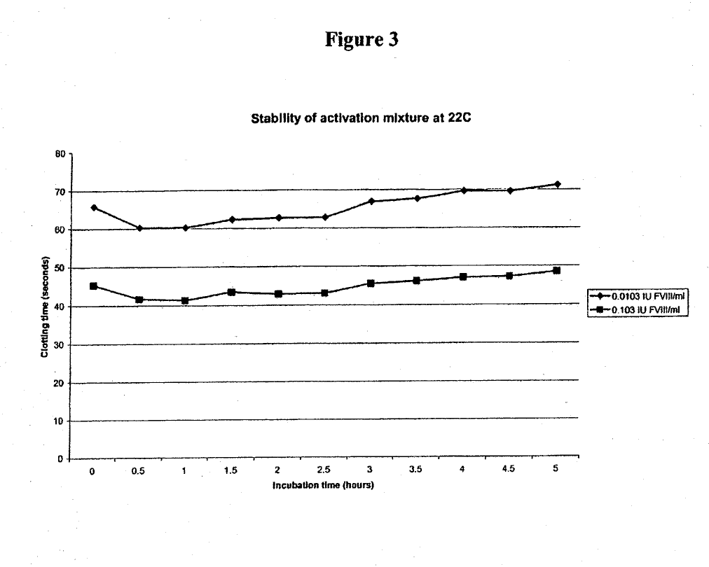

A graph illustrating the stability of the activation mixture at 22° C. over 5 hours.

FIG. 4

A graph illustrating the dose response for FVIII added to FVIII-deficient plasma. The graph presents the clotting time (seconds) (vertical axis) and the concentration of FVIII spiked in the FVIII-Deficient plasma (horizontal axis).

### DETAILED DESCRIPTION OF THE INVENTION

### Activation Mixture

As described herein, the inventors have discovered that an activation mixture can be used to replace the individual components (i.e. factor-deficient substrate plasma, an activator, and a phospholipid or factor-deficient substrate plasma and an activator/phospholipid) that are used in the existing one stage assay method.

An “activation mixture” in the context of the present invention refers to factor-deficient substrate plasma, an activator, and a phospholipid that is mixed together before it is contacted with a test sample. Accordingly, the activation mixture is prepared before it is used in a reaction containing a test sample—such as in an assay method for a blood coagulation factor:

In a preferred embodiment, the present invention relates to an activation mixture comprising factor-deficient substrate plasma, an activator, and a phospholipid.

In another preferred embodiment, the present invention relates to an activation mixture comprising or consisting essentially of factor-deficient substrate plasma, an activator, and a phospholipid.

Typically, the activation mixture is prepared by mixing substantially equal volumes of the factor-deficient substrate plasma, activator, and phospholipid together to form a single reagent.

Preferably, the activation mixture is pre-heated before it is used in the assay method of the present invention. More preferably, the activation mixture is pre-heated to about 37° C.

In a preferred embodiment of the present invention, the activation is pre-incubated at about 37° C. for 10 minutes before it is used in the assay method of the present invention.

Two of the components may be provided as a single reagent which is then added to the third component to form the activation mixture.

Preferably, the activator and phospholipid are provided as a single reagent which is then mixed with a substantially equal volume of factor-deficient substrate plasma.

Commercially available mixtures of activator and phospholipid are generally referred to as APTT reagents, which have been extensively reviewed in Poller and Thomson (1972) J. Clin, Pathol. 25, 1038-1044.

By way of example, APTT reagents are available from various commercial sources that include, but are not limited to, bioMerieux, France; Sigma Diagnostics, USA; Helena Haemostasis Systems Ltd, UK; and Instrumentation Laboratory, USA.

In a preferred embodiment, the APTT reagent used is the Instrumentation Laboratory APTT-SP (liquid) (Catalogue number 20006300) which comprises a silica activator and a phospholipid mixture.

Preferably, the activation mixture is stable at 4° C. and/or 22° C. for at least 5 hours.

Advantageously, the stability may also avoid temporal drift during the assay.

### Blood Coagulation Factor

As used herein, the term “blood coagulation factor” refers to any blood coagulation/blood clotting factor that is involved in or associated with the prevention of blood loss at a site of injury or damage, for example, at a wound.

Blood coagulation involves the formation of a semisolid mass of material, the blood clot, which plugs the wound. The clot consists of aggregated platelets and a mesh of fibrin molecules which include a number of plasma proteins, at least one tissue protein, phospholipid membrane surfaces, calcium ions and platelets. The mechanism of blood coagulation, and the components involved are comprehensively described in several review articles including Cell 53 (1988) 505-518; Biochem. 30 (1991) 10363-10379; Methods of Enzymatic Analysis, Vol. V, chapter 3, 3rd ed., Academic Press, New York (1983).

The proteins that are involved in the blood clotting process are commonly referred to as factors.

Blood coagulation factors include Factor H, Factor V, Factor VII, Factor VIII, Factor IX, Factor X, Factor XI, Factor XII and Factor XIII. Reference to a factor by its number identifies the corresponding protein to a person skilled in the art.

Preferably, the blood coagulation factor in the context of the present invention refers to any one of Factor VIII, Factor IX or Factor XI. Most preferably, the blood coagulation factor is Factor VIII.

Therefore, in a highly preferred embodiment of the present invention, the blood coagulation factor is Factor VIII.

### Factor-Deficient Substrate Plasma

A person skilled in the art will appreciate that the factor-deficient substrate plasma that is used will be the same as the blood coagulation factor that is being assayed, Thus, by way of example, if the blood coagulation factor to be assayed is Factor VIII, Factor IX or Factor XI then Factor VIII-, Factor IX-, or Factor XI-deficient substrate plasma will be used, respectively.

The factor-deficient substrate plasma that is used in the assay method of the present invention can be obtained from various sources.

The preparation of the plasma may include measures to render it cell free (double centrifugation), to freeze it rapidly and to stabilise it with a buffer when freeze-drying is applied (Godfrey et al. (1975) Thromb. Diath. Haemorrh. 34, 879-882).

The substrate plasma may be obtained from severe haemophilia patients—such as haemophilia A patients. However, because prophylactic treatment for severe haemophiliacs is becoming more intense, it is more difficult to obtain plasma not containing the blood coagulation factor. Moreover, the frequency of HCV infection is also problematic.

An alternative source is to purchase substrate plasma from commercial sources—such as those sources described in Barrowcliffe et al. (1981) Haemostasis 11, 96-101.

Another possibility is to use artificial blood coagulation factor-deficient plasma obtained by the selective removal of the blood coagulation factor from normal plasma. This may be done by physical, chemical, or immunological treatment.

Suitably, the residual blood coagulation factor coagulant activity of the substrate plasma should be as low as possible, typically less than 1% of normal.

Also, antibodies to the blood coagulation factor should be absent while other clotting factors should be present in concentrations sufficiently high in order not to be rate-limiting. A large difference should be present between the blank coagulation time and the coagulation time at high blood coagulation factor concentrations, usually implying a steep slope with linearity (after appropriate transformation) over a wide rage of blood coagulation factor concentrations.

Preferably, artificial blood coagulation factor-deficient plasma—such as artificial Factor VIII deficient plasma (Biomerieux/Organon Teknika Corp, USA) is used. This plasma is chemically depleted and contains normal vWF levels.

### Activator

Typically, the activator that is used is an activator of Factor XII, A wide variety of soluble and insoluble activators of Factor XII are known.

Factor XII is the circulating precursor of the proteinase Factor XIIa which activates Factor XI. Factor XI is the circulating precursor of Factor XIa, which converts Factor IX into Factor IXa. Factor IXa and Factor VIII participate together in the activation of Factor X in the intrinsic pathway of coagulation.

Generally, the activators have in common a net negative charge on either a surface—such as kaolin, celite, glass, ellagic acid and micronized silica, or on a component of high molecular weight, like dextransulfate.

A person skilled in the art will appreciate that the activators described herein are not exhaustive and that others may be used.

Typically, kaolin, ellagic acid or micronized silica are used in the one stage assay. Ellagic acid and micronized silica may be used as an activator when coagulation times are recorded in a coagulomoter with photooptical clot detection; kaolin is the most widely used in other coagulomoters (Barrowcliffe et al., 1981).

In a preferred embodiment, the activator is micronized silica.

The activator may also comprise Factor IXa. Factor LXa is a serine protease that activates Factor X in the intrinsic pathway of coagulation and converts Factor IX into Factor IXa.

The concentration of activator should be chosen such that coagulation times are as short as possible after applying the optimal activation time.

### Phospholipid

Generally, the best source of phospholipid for in vitro tests are platelets themselves in the form of platelet-rich plasma obtained from a severe haemophilic (Nilsson et al. (1957) Acta. Med. Scand. 159, 35-57 (1957)). However, this is not a practical approach for the majority of laboratories and other reagents have to be used.

A mixture of a negatively charged phospholipid—such as phosphatidylserine—and an uncharged phospholipid—such as phosphatidylcholine—may serve as a suitable reagent (Zwaal & Hemker (1982) Haemostasis 11, 12-39).

Phospholipid extracts of different sources may be used—such as bovine, rabbit or human brain, as described in Bell and Alton (1954) Nature 174, 880-881; Hjort et al. (1955) J. Lab. Clin. Med. 46, 89-97; and Barrowcliffe et al. (1982) Homeostasis 11, 96-101).

The phospholipids may be substantially purified phospholipid.

In a preferred embodiment of the present invention, synthetic phospholipid is used, for example, substantially purified synthetic phospholipid.

Advantageously, a phospholipid concentration is used that yields the shortest coagulation time to ensure that small variations in phospholipid concentration in the test system (due to differences in phospholipid content of test samples) have minimal effect on the coagulation times. Sub- and supra-optimal concentrations result in longer coagulation times.

### Calcium Reagent

To begin the blood coagulation factor dependent part of the coagulation cascade, calcium ions must be added in an amount that the citrate (which is present as the anticoagulant of the substrate plasma) that is present in the mixture is overcome and an optimal free calcium concentration is obtained.

Any chemical source of calcium cation may be used. For example, the source of calcium cation (Ca⁺⁺) may be CaCl₂, Ca(NO₂)₂, CaSO₄, or other inorganic calcium to cation containing compounds may be used.

Preferably, the source of calcium cation is CaCl₂.

Typically, 25 to 33 mM calcium reagent is used in a volume equivalent to that of substrate plasma. However, a person skilled will be able to determine the optimal concentration for the calcium reagent by establishing the shortest clotting time (Lenahan & Philips (1966) Clin. Chem. 12, 269-273).

Preferably, the calcium regent is pre-heated before it is used in an assay method according to the present invention. More preferably, the calcium regent is pre-heated to about 37° C.

### Assay Method

In a further aspect, the present invention relates to an assay method for determining the amount of blood coagulation factor in a test sample.

The assay method according to this aspect of the present invention comprises the step of adding an activation mixture comprising factor-deficient substrate plasma, activator, and phospholipid.

Advantageously, the assay method described herein is faster to perform than existing methods. The reasons for this are two fold. Firstly, the assay method described herein comprises one less step than the existing one stage assay which requires that the blood coagulation factor-deficient plasma is added to the activator and phospholipid or a mixture thereof. As described herein, the factor-deficient plasma, activator and phospholipid are provided as a single mixture before they are used in an assay. Secondly, the 10 minute incubation step at about 37° C. is omitted. Instead the activation mixture is pre-heated to the required temperature and so it can be mixed directly with other assay components.

Advantageously, the assay method described herein is simpler to perform than existing methods. This is by virtue of the assay method of the present invention comprising fewer steps, as described above.

Advantageously, the assay method described herein may be easier to automate than existing methods. This is because the assay method of the present invention comprises fewer steps and the 10 minute incubation step at about 37° C. is not required. Accordingly, the components of the assay method described herein can be added directly to the assay mix and the clotting time/clotting end point measured.

In a preferred embodiment, the assay method comprises the steps of: (a) providing a test sample; (b) providing an activation mixture according to the present invention; (c) adding together the test sample and the activation mixture; (d) adding a calcium reagent; and (d) measuring the clotting time/clotting end point.

In performing the assay of the present invention, variations in protein concentrations, incubation times, reagent concentrations, and temperatures may be employed. The selection of particular assay parameters will be influenced by the source, type, and size of the sample to be assayed, the anticipated levels of blood coagulation factor contained in the test sample, and the sensitivity desired. Taking these circumstances into account, selection of assay parameters will be apparent to those skilled in the art.

Media that are used for the dilution of standard and test samples generally contain a buffering agent that does not contain calcium ions—such as, but not limited to, barbital, barbital-acetate, imidazole or Tris.

The buffering agents are adjusted to physiological pH and supplemented with saline for providing physiological ionic strength.

If materials differing in protein concentration are tested—such as concentrate against plasma—additional protein may be included in the buffer to mask such differences. Alternatively, the first dilution of a concentrate (pre-dilution) may be preformed in blood coagulation factor-deficient plasma to mimic a normal plasma sample.

A reaction temperature of about 37° C. is commonly used both for the activation of blood coagulation factors and for the coagulation steps. However, a person skilled in the art will understand that the temperature may be other than 37° C., since at lower temperatures, for example, 30, 31, 32, 33, 34, 35 or 36° C., to slightly higher temperatures, for example, 38, 39 or 40° C., the clotting times/clotting end point may be affected. Such an affect, may cause the clotting times to be increased or decreased.

However, irrespective of whether the temperature is higher or lower than 37° C., it is desirable to maintain a constant temperature throughout the assay. This is because slight variations during the assay may influence the clotting time/clotting end point.

Various devices—such as tubes and reaction cuvettes—are typically used in the assay method of the present invention. Such devices may be made of, for example, plastic or glass. The composition of the devices is of little consequence because contact activation in the assay system is standardised by the addition of the activation mixture.

Preferably, for use in machines—such as semi-automated or fully automated machines (e.g. coagulometers)—the devices should be optically regular (Zacharski & Resenstein (1978) Am. J. Clin. Pathol. 70, 280-286).

In performing the assay method described herein, a person skilled in the art will understand that the dilutions used should be chosen so that after appropriate transformation a straight line can be drawn though a number of points typically derived from a minimum of 3 different dilutions. Generally, either transformation of both the dose (blood coagulation factor concentration) and the response (coagulation time) to their logarithms, or transformation of the dose only, typically results in a straight line for a range of dilutions.

Generally, the sequence of adding the reagents is not an important factor. However, for reasons of stability, it is better not to start with the blood coagulation factor test sample—such as the diluted blood coagulation factor test sample—but with the activation mixture, followed by the blood coagulation factor test sample. The calcium reagent is then added to the activation mixture/test sample.

### Test Sample

The term “test sample” as used herein, has its natural meaning.

A test sample may be any physical entity in which the amount of blood coagulation factor in the sample is determined according to the present invention.

The sample may be or may be derived from a mammal. Preferably, the test sample is or is derived from an animal or a human. Most preferably, the test sample is or is derived from a human.

The sample may be or may be derived from the expression of normal or modified human genes encoding blood coagulation factors in mammalian or non-mammalian expression systems.

The sample may be or may be derived from biological material, including recombinant biological material.

The test sample may be or may be derived from blood or a component thereof—for example, plasma—such as venous or capillary plasma.

Preferably, the test sample is pre-heated before it is used in an assay method according to the present invention. More preferably, the test sample is pre-heated to about 37° C.

Blood may be prepared according to the methods described by Langdell et al. (1953) J. Lab. Clin. Med. 41; 637-647). Briefly, blood is obtained by venepuncture—such as antecubital or jugular venepuncture—and is mixed immediately with anticoagulant—such as sodium citrate or sodium oxalate

Suitably, the plasma sample should be stored on melting ice until assayed and usually, within 1 hour after drawing the blood sample.

Venous plasma may be prepared according to the method of Hardisty and Macpherson (1962) Thromb. Diath. Haemorrh 7, 215-229. Briefly, 9 volumes of blood are taken from an antecubital vein into 1 volume of 3.1% trisodium citrate and centrifuged at about 2000 g for at least 15 minutes to obtain platelet poor plasma. The plasma may be stored at −20° C. or below and thawed immediately before use. Test and control plasmas for assay are diluted with 9 volumes of veronal-buffered isotonic saline (VBIS) at pH 7.35 containing 0.15% trisodium citrate. Further dilutions can be made in VBIS without citrate.

Capillary plasma may be prepared according to the method of Hardisty and Macpherson (1962) Thromb. Diath. Haemorrh 7, 215-229. Briefly, 0.2 ml of free flowing capillary blood is taken into 1.8 ml of VBIS containing 0.2% trisodium citrate to give a 1:10 dilution of whole blood, as described by Dormandy and Hardisty (1961) J. Clin. Path. 14, 543, and centrifuged at 2000 g for 15 minutes, to obtain dilute platelet-poor Plasma. Further dilutions can be made in VBIS. If a low blood coagulation factor concentration is expected, a further 0.2 ml of capillary blood may be taken into 0.8 ml of VBIS containing 0.22% trisodium citrate, to give 1:5 dilution of whole blood

### Blood Coagulation Factor Standard

Another parameter in the assay method described herein, is the blood coagulation factor standard. Generally, the standard should be similar in nature to the test sample. This decreases the risk of nonparallelism, eliminates the effects exerted by protein in the dilution medium, and yields more reproducible results with less variation.

One method of preparing a plasma standard is described by Harper & Chauhan (1981) Am. J. Clin. Pathol. 77, 614-618. Briefly, a plasma pool from a limited number of donations—such as four—is rapidly frozen in small aliquots and stored either frozen or lyophilised. A provisional potency of 1 U/ml (where a unit is defined as that amount of blood coagulation factor that is present in 1 ml of normal plasma) is then assumed for this standard. The blood coagulation factor content of fresh plasma samples is then measured against the standard until approximately 30-40 results are obtained. Because the mean result for these samples is by definition, 1 U/ml, the real potency of the standard can then be calculated by dividing 1 U/ml by the apparent mean of the plasma.

A person skilled in the art will appreciate that such methods have now been superseded by the introduction of International Standards for blood coagulation factors—such as Factor VIII—in Concentrate and Plasma which define the International Unit (IU).

An international standard for blood coagulation factors—such as Factor VIII—has been established by the World Health Organisation (Bangham et al. (1971) Bull. Wld. Hlth. Org. 45, 337-351) and since this time various international standards have been devised. The different versions of the International Concentrate standards have consisted of intermediate, high and very high purity plasma-derived concentrates. The current version (6^{th} IS) is a recombinant blood coagulation factor.

These standards may be obtained from the National Institute of Biological Standards and Control, London, Potters Bar, UK. For example, the 21st British Standard FVIII Plasma (NIBSC code 00/586) may be used.

### Measuring the Clotting Time/Clotting End Point.

Various methods may be used for clot detection.

By way of example, a hook or tilting tubes in a water bath may be used for manual determination. Alternatively, semi-automated coagulometers may be used for detecting fibrin thread formation, an increase in viscosity, a displacement of steel rods or balls by the fibrin clot, a decrease in light transmission of the reaction mixture when the clot forms (using photo-optical instruments) or other types of instruments based on changes in transmission of light beams.

Automated machines may also be used—such as semi- or fully-automated coagulometers for detection of the endpoint (Harms et al. (1978) Am. J. Clin. Pathol. 70, 560-562).

Generally, coagulometers provide a more rapid method of testing.

Advantageously, the clotting time/clotting end point may be measured using near-patient testing or point-of-care devices.

A person skilled in the art will understand that once the clotting time/clotting end point has been measured or determined, various calculation and statistical analysis methods may then be applied to determine the amount of blood coagulation factor in a test sample.

Preferably, the method that is used is programmed calculation based on regression analysis because it will establish whether an assay is valid and it will also provide a more accurate estimate of the potency, not biased by, for example, errors in subjectively drawing dose response curves by hand.

Typically, an assay will be considered as invalid when the dose-response curves deviate from linearity or from parallelism. The presence of such deviations can be assessed by means of analysis of variance. To this end, it is necessary to test replicates for each dilution to determine the random error, and to test every preparation in at least three dilutions. Computer programs to analyse parallel-line bioassays have been described by Williams et al. (1975) Brit. J. Haematol. 31, 13-23; Counts & Hays (1979) Am. J. Clin. Pathol. 71, 167-171; Kirkwood & Snape (1980) Clin. Lab. Haematol (1980) 2, 155-167.

### Kit

In a further aspect, the present invention relates to a kit for determining the amount of blood coagulation factor in a test sample comprising a vessel containing an activation mixture.

An additional vessel may be included containing or comprising diluent.

An additional vessel may be included containing or comprising a calcium reagent.

An additional vessel may be included containing or comprising blood coagulation factor such as a blood coagulation factor standard.

### Point of Care & Near Patient Testing

Advantageously, the clotting time and/or clotting end point may be measured using a point-of-care testing device in which, for example, whole blood or plasma is added to a component of the device—such as a cartridge or cassette—containing the modified activation mixture described herein.

Point-of-care testing offers the advantage of providing immediate results, in contrast to conventional testing, where there is a waiting period, that could be anywhere from several hours to weeks, during which the specimens are transported to a laboratory testing facility, processed, and results dispatched.

Point of care testing may be performed rapidly and on site, such as in a doctor's office, at a bedside, in a laboratory—such as clinical laboratories hospital, in the field or other such locations.

A review of point of care testing of hemostasis is presented in Thrombosis Journal (2003) 1, 1. As described therein, various methods and devices for measuring blood clotting time/clotting end point have been devised. Moreover, other bedside instruments which evaluate blood viscoelastic properties and/or platelet function allow further information about fibrinolysis and platelet function to be rapidly obtained.

Most of the instruments currently available can perform multiple coagulation tests, depending on which cartridge or test tube is selected. Examples of such instruments are outlined below.

The Hemochron automated instruments (International Technidyne Corp, USA-ITC) J (Extra Corpor Technol 1999, 31:130-134) includes two types of devices employing tubes containing celite or kaolin, or cartridges preloaded with a preparation of silica, kaolin and phospholipid.

The Automated Coagulation Timer II (ACT II) and Hepcon Hemostasis Management System (HMS) (Medtronic Hemotec, USA) measure blood clotting using kaolin or, less commonly, celite as activator.

Heparin management Test (HMT), performed with Rapidpoint Coag machine (Bayer, USA), uses disposable test cards containing a reaction chamber with test-specific reagents (celite and stabilizers) and paramagnetic iron oxide particles (PIOPs) which move in response to an oscillating magnetic field.

i-STAT analyzer (Abbott, USA) is designed for whole-blood-based testing with celite preloaded cartridges.

The Actalyke Activated Clotting Time (Array Medical, Somerville, N.J.) test system employs electromagnetic clot detection such as Hemochron series. In addition to celite, this system performs MAX-ACT, which is a new type of ACT which uses tubes containing a “cocktail” of activators (celite, kaolin and glass beads) to maximally convert all Factor XII to XIIa.

A variety of automated systems for evaluating blood clotting formation are also available (Blood Coag Fibrinol 2001, 12:327-337; Br J Anaesth 1995, 75:771-776).

The TEG device consists of a bench-top instrument comprising a dual-channel Coagulation Analyser (Teg^{R}—Hemoscope, USA) and a Teg^{R} analytical software. TEG gives a graphic representation of aspects of clot formation and lysis.

The ROTEG analysis (Pentapharm, GMBH) is based on rotation thromboelastography, which is related to, but in some aspects different from classical analysis with TEG.

The Sonoclot analyzer (Sienco Inc, USA) measures changes in the viscoelastic properties of blood clot.

The Platelet function Analyzer-100 (PFA-100, DADE Behring, USA) assesses whole blood platelet function by measuring the closure time (CT) required for platelets in citrated whole blood to occlude a precisely defined aperture cut into a synthetic membrane coated with either collagen and epinephrine or collagen and adenosine diphosphate.

As described herein, the amount of blood coagulation factor in the test sample may then be calculated from the blood clotting time/clotting end point.

Advantageously, the clotting time/clotting end point may also be measured using near patient testing.

The use of near patient testing devices is reviewed in British Journal of Haematology (2001) 113, 847-852.

In general, near patient testing devices are small portable instruments able to detect clot formation upon the addition of, for example, whole blood—such as citrated blood or plasma—to a reaction chamber built in a small cartridge that incorporates reagents—such as freeze-dried reagents—with or without calcium chloride.

In the context of the present invention, such cartridges may contain the modified activation mixture.

In one type of near-patient testing device, the test material is added within the reactive area of the cartridge, typically pre-warmed to 37° C., and the reaction is started by reconstituting the reagent. The time elapsed from the beginning of the reaction is recorded and displayed by the device as a clotting time/clotting end point.

In other types of near-patient testing device, paramagnetic iron oxide particles are mixed with the reagent within the reaction chamber. The addition of blood starts the reaction and paramagnetic particles are free to move within the reaction chamber as an electromagnet turns on and off. Movement stops as the clot forms and this is recorded by the device as a clotting time/clotting end point.

As described herein, the amount of blood coagulation factor in the test sample may then be calculated from the blood clotting time/clotting end point.

Other devices include, but are not limited to Coagucheck (Roche Diagnostics); Protime Microcoagulation System (International Technidyne Corp). Other devices are described at: www.pointofcare.net/vendors/index.htm.

The invention will now be further described by way of Examples, which are meant to serve to assist one of ordinary skill in the art in carrying out the invention and are not intended in any way to limit the scope of the invention.

### EXAMPLES

### Example 1

### Comparison of Established and Modified Methods for the One-Stage Clotting Assay

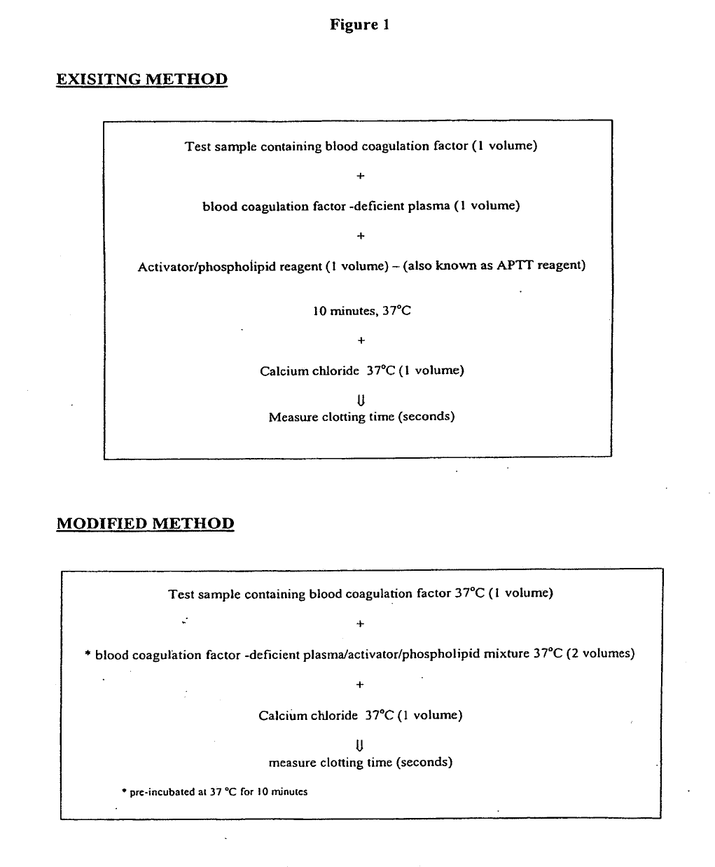

A direct comparison of the established and modified methods (FIG. 1) has been performed using a test plasma sample with low FVIII:C (approx 25% normal). Estimation of FVIII:C in this test sample was calculated relative to the 21st British Standard FV111 Plasma.

### Materials and Methods

### Assay Samples

Reference Standard: 21st British Standard FVIII Plasma (NIBSC code 00/586) with an assigned value of 0.55 IU per ml

Test sample: Normal human plasma diluted in FVIII-deficient plasma to give a FVIII:C content of approx 0.25 IU per ml. Stored as frozen aliquots

### Instrument:

KC-4 coagulometer (Amelung).

### Method

Two independent assays were carried out using each method according to the balanced design (12-place assay):

Standard/Test where three dilutions of both Standard and Test are assayed in replicate within each assay. (Standard is diluted 1/10, 1/30, 1/100 and test is diluted 1/3, 1/10, 1/30)

i. Established Method (KC-4 Coagulometers)

| | |
|---|---|
| FVIII-deficient plasma (Organon Teknika/Biomerieux) + | 0.1 ml |
| Standard/Test dilutions + | 0.1 ml |
| APTT reagent (Instrumentation Laboratory APTT-SP liquid) | 0.1 ml |
| Incubate at 37° C. for 10 mins | |
| CaCl₂ 25 mmol/L | 0.1 ml |
| Measure clotting time | |

ii. Modified Method (According to the Present Invention)

| | |
|---|---|
| Standard/Test dilutions + | 0.1 ml (37° C.) |
| Pre-incubated FVIII-deficient plasma/APTT reagent* + | 0.2 ml (37° C.) |
| CaCl₂ 25 mmol/L | 0.1 ml (37° C.) |
| Measure clotting time | |

*incubate 1 volume of FVIII-deficient plasma (Organon Teknika/Biomerieux) with 1 volume of APTT reagent (IL APTT-SP liquid) for 10 mins at 37° C.

### Note on Analysis

Relative potency estimates were analysed according to parallel line bio-assay principles relating log-dose to log response as described in A. D. Curtis “The Statistical Evaluation of Factor VIII Clotting Assays” in Scand. J. Haematol. supplement (1984) 41, 33 p 55-68. This analysis relies on the parallelism of the dose-response relationships of standard and test samples. This criteria was satisfied for all assays:

Slope

| Method | Assay | Standard | Test sample |
|---|---|---|---|
| Established | 1 | −0.1525 | −0.1756 |
| | 2 | −0.1582 | −0.1755 |
| Modified | 1 | −0.1471 | −0.1514 |
| | 2 | −0.1482 | −0.1515 |

### Results

i. Established Method

### a) Clotting Time (Seconds)

| | | | |
|---|---|---|---|
| Sample | Dilutions | Assay 1 | Assay 2 |

| | | | | | |
|---|---|---|---|---|---|
| Standard | 1/10 | 43.0 | 42.8 | 42.6 | 42.7 |
| | 1/30 | 52.0 | 52.2 | 50.2 | 51.4 |
| | 1/100 | 60.9 | 61.1 | 61.3 | 61.5 |
| Test plasma | 1/3 | 39.0 | 39.9 | 39.0 | 39.7 |
| | 1/10 | 50.3 | 50.9 | 50.9 | 49.4 |
| | 1/30 | 59.4 | 58.7 | 59.5 | 58.3 |

### b) Relative Potency Estimates (IU Per ml)

| Assay | Mean potency | 95% confidence limits |
|---|---|---|
| 1 | 0.23 | 0.21-0.25 |
| 2 | 0.22 | 0.20-0.25 |

ii. Modified Method

### a) Clotting Time (Seconds)

| | | | |
|---|---|---|---|
| Sample | Dilutions | Assay 1 | Assay 2 |

| | | | | | |
|---|---|---|---|---|---|
| Standard | 1/10 | 47.4 | 47.8 | 48.1 | 48.5 |
| | 1/30 | 56.2 | 56.6 | 56.9 | 57.4 |
| | 1/100 | 66.7 | 66.9 | 67.2 | 68.7 |
| Test plasma | 1/3 | 44.5 | 45.0 | 44.8 | 45.9 |
| | 1/10 | 55.1 | 53.9 | 55.9 | 54.9 |
| | 1/30 | 64.6 | 62.2 | 64.3 | 64.2 |

### b) Relative Potency Estimates (IU Per ml)

| Assay | Mean potency | 95% confidence limits |
|---|---|---|
| 1 | 0.24 | 0.21-0.27 |
| 2 | 0.24 | 0.20-0.27 |

### CONCLUSION

Potency estimates obtained using the modified method were not significantly different to the estimates obtained using the established method.

### Example 2

### Stability of the Activation Mixture

Instrumentation Laboratory APTT reagent (APTT-SP) (silica activator+phospholipid mixture) is mixed with an equal volume of Factor VIII-deficient plasma (Organon Teknica) and incubated at 37° C. for 10 mins. The mixture is then stored at 4° C. or 22° C.

Clotting time is measured after mixing 0.2 ml of pre-activated reagent mixture (pre-warmed to 37° C.). with 0.2 ml of Factor VIII/calcium chloride mixture (pre-warmed to 37° C.). Measurements are taken every 30 minutes using fresh aliquots of Factor VIII test sample (to avoid the effect of Factor VIII instability).

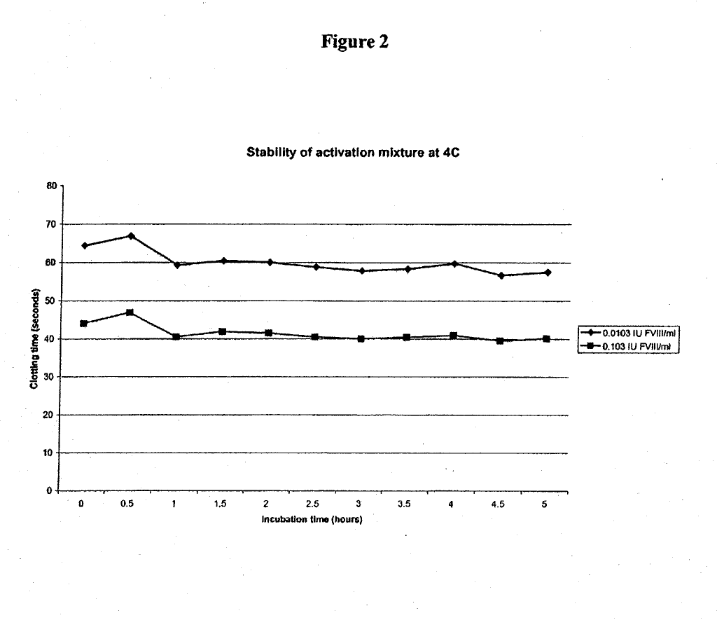

The results are shows in FIGS. 1 and 2.

Thus, it is demonstrated that the activation mixture is stable at 4° C. and 22° C. for at least 5 hours.

### Example 3

### Sensitivity of the Modified Method

Factor VIII-deficient plasma spiked with varying concentrations of purified FVIII (1 volume) was mixed with pre-activated reagent prepared as in Example 1 (2 volumes) and calcium chloride (25 mmol/l) (1 volume) and the clotting time measured.

All reagents were pre-warmed to 37° C.

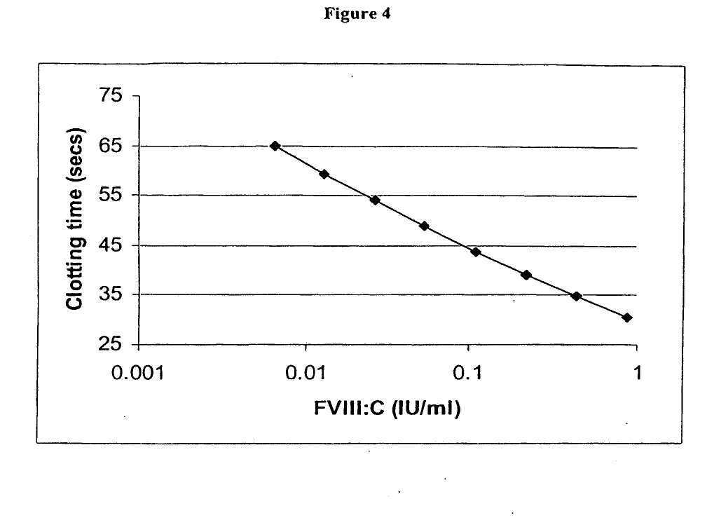

The results are shown in FIG. 4.

The graph presents the clotting time (seconds) (vertical axis) and the concentration of FVIII spiked in the FVIII-Deficient plasma (horizontal axis).

It follows that the modified method described herein is suitable for use in NPT/POC devices.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in biology or related fields are intended to be within the scope of the following claims.

The invention will now be further described by the following numbered paragraphs:

1. An activation mixture comprising factor-deficient substrate plasma, an activator, and a phospholipid.

2. An activation mixture according to paragraph 1 wherein the activator is micronised silica.

3. An activation mixture according to paragraph 1 where the phospholipid is synthetic phospholipid.

4. An activation mixture according to any one of paragraphs 1 to 3 wherein the activator and phospholipid are provided as a single reagent.

5. An activation mixture according to paragraph 4 wherein the single reagent is APTT reagent.

6. An activation mixture according to any one of the preceding paragraphs wherein the factor-deficient substrate plasma is chemically depleted or immuno depleted.

7. An activation mixture according to any one of the preceding paragraphs wherein the activation mixture is stable at 4° C. and/or 22° C. for at least 5 hours.

8. A method for preparing an activation mixture according to any one of paragraphs 1 to 7 comprising the step of mixing together factor-deficient substrate plasma, an activator and a phospholipid.

9. A method according to paragraph 8, wherein the activator and phospholipid are provided as a single reagent.

10. A method according to paragraph 9, wherein the single reagent is APTT reagent.

11. An assay method for determining the amount of blood coagulation factor in a test sample comprising the step of adding an activation mixture according to any one of paragraphs 1 to 7 to the test sample.

12. An assay method according to paragraph 11 comprising the steps of:

(a) providing a test sample;
(b) providing an activation mixture according to any one of paragraphs 1 to 7;
(c) adding together the test sample and the activation mixture;
(d) adding a calcium reagent;
(e) measuring the clotting time/clotting end point; and
(f) determining the amount of blood coagulation factor in the test sample.

13. An assay method according to paragraph 11 or 12 wherein each of the assay components recited in steps (a), (b) and (d) are pre-heated.

14. An assay method according to paragraph 13 wherein each of the assay components recited in steps (a), (b) and (d) are pre-heated to about 37° C.

15. An assay method according to any one of paragraphs 11 to 14, wherein the clotting time/clotting end point is measured using a coagulometer.

16. An assay method according to any one of paragraphs 11 to 14, wherein the clotting time/clotting end point is measured using a point of care testing device and/or near patient care device.

17. A kit for determining the amount of blood coagulation factor in a test sample comprising a first vessel containing an activation mixture according to any one of paragraphs 1 to 7.

18. A kit according to paragraph 16, wherein the kit comprises an additional vessel containing a calcium reagent.

19. A kit according to paragraph 17 or paragraph 18 wherein the kit comprises an additional vessel containing a blood coagulation factor.

20. A kit according to any of paragraphs paragraph 17 to 19 wherein the kit comprises a point of care testing device or near patient care device for measuring the clotting time/clotting end point.

21. Use of an activation mixture according to any one of paragraphs 1 to 7 in an assay method for determining the amount of blood coagulation factor in a sample.

22. The use according to paragraph 21 wherein the activation mixture is preheated.

23. The use according to paragraph 22 wherein the activation mixture is preheated to about 37° C.

1-10. (canceled)

11. An assay method for determining the amount of blood coagulation factor in a test sample comprising adding an activation mixture to the test sample, wherein the activation mixture comprises factor-deficient substrate plasma, an activator, and a phospholipid.

12. An assay method according to claim 11 comprising:
(a) providing a test sample;
(b) providing an activation mixture comprising factor-deficient substrate plasma, an activator, and a phospholipid;
(c) adding together the test sample and the activation mixture;
(d) adding a calcium reagent;
(e) measuring the clotting time/clotting end point; and
(f) determining the amount of blood coagulation factor in the test sample.

13. An assay method according to claim 12 wherein each of the assay components recited in (a), (b) and (d) are pre-heated.

14. An assay method according to claim 13 wherein each of the assay components recited in (a), (b) and (d) are pre-heated to about 37° C.

15. An assay method according to claim 11, wherein the clotting time/clotting end point is measured using a coagulometer.

16. An assay method according to claim 11, wherein the clotting time/clotting end point is measured using a point of care testing device and/or near patient care device.

17-18. (canceled)

19. A method of using an activation mixture in an assay method for determining the amount of blood coagulation factor in a sample, wherein the activation mixture comprises factor-deficient substrate plasma, an activator, and a phospholipid, and wherein the activation mixture is optionally preheated.

20. The method according to claim 19 wherein the activation mixture is preheated to about 37° C.
